(19) United States
(12) Patent Application Publication
(10) Pub. No.: US 2013/0243702 A1
(43) Pub. Date: Sep. 19, 2013

(54) Mouthwash Composition for Managing Oral Mucositis, Process and Methods Thereof

(71) Applicants: Divya Ravindran, Thiruvananthapuram (IN); Ravikumar Rejnish Kumar, Thiruvananthapuram (IN); Kunnambath Ramadas, Thiruvananthapuram (IN); Madhavan Radhakrishna Pillai, Thiruvananthapuram (IN)

(72) Inventors: Divya Ravindran, Thiruvananthapuram (IN); Ravikumar Rejnish Kumar, Thiruvananthapuram (IN); Kunnambath Ramadas, Thiruvananthapuram (IN); Madhavan Radhakrishna Pillai, Thiruvananthapuram (IN)

(21) Appl. No.: 13/883,551

(22) PCT Filed: Nov. 3, 2011
(86) PCT No.: PCT/IB11/54877

§ 371 Date: May 3, 2013

(30) Foreign Application Priority Data
Nov. 4, 2010 (IN) 3310/CHE/2010

(51) Int. Cl. A61K 8/97; A61Q 11/00

(52) U.S. Cl. CPC A61K 8/97; A61Q 11/00
USPC 424/58

(57) ABSTRACT

The present disclosure is in relation to a herbal cleansing composition comprising extracts from five plants namely Emblica officinalis, Terminalia chebula, Terminalia bellerica, Glycyrrhiza glabra and Azadiracta indica. Further, the present disclosure also relates to a process of obtaining said composition and applications thereof, wherein the composition is preferably used as an oral wash for delaying the onset of oral mucositis in cancer patients undergoing radiation treatment.

### TECHNICAL FIELD

The present disclosure is in relation to a herbal cleansing composition comprising extracts from five plants namely, Emblica officinalis, Terminalia chebula, Terminalia bellerica, Glycyrrhiza glabra and Azadiracta indica. The herbal cleansing composition is used as an oral wash for delaying the onset of oral mucositis in cancer patients undergoing radiotherapy treatment.

### BACKGROUND

Oral mucositis is a complex biological process, involving direct damage to the dividing cell of the oral epithelium with depletion of the basal epithelium modulated by the immune system, the inflammatory process and by super added infection by the oral bacterial flora, especially the aerobic gram negative bacteria. Oral bacteria colonize in the denuded connective tissues and their cell wall components activate macrophages to produce additional inflammatory cytokines in patients undergoing radiotherapy for Head and Neck cancers and this is the dose limiting toxicity and patients may discontinue treatment due to this.

Considerable variation exists in the management of oral mucositis. Maintaining good oral hygiene and frequently rinsing the mouth with saline and sodium bicarbonate solution is generally recommended. Following the onset of oral mucositis, the management is mainly supportive; consisting of alleviation of pain, control of secondary infection and maintaining adequate hydration and nutrition. Given the profound possibility of oral mucositis and its importance as a dose-limiting factor in head and neck radiotherapy, prophylaxis of this acute toxicity is highly desirable. Several agents have been tried to minimize the severity of oral mucositis. Several molecules such as amifostine, benzydamine, calciumphosphate, honey, hydrolytic enzymes, ice chips, isegananm oral care and zinc sulphate, GCSF are found to have been tried in preventing or reducing the severity of radiation/chemotherapy induced mucositis with varying results. But so far no method has been found to be effective.

### STATEMENT OF DISCLOSURE

Accordingly the present disclosure provides a composition comprising extracts of Emblica officinalis, Terminalia chebula, Terminalia bellerica, Glycyrrhiza glabra and Azadiracta indica; a method for obtaining a composition comprising extracts of Emblica officinalis, Terminalia chebula, Terminalia bellerica, Glycyrrhiza glabra and Azadiracta indica, said method comprising acts of—a) soaking plant parts of Emblica officinalis, Terminalia chebula, Terminalia bellerica, Glycyrrhiza glabra and Azadiracta indica in a solvent to obtain extract of the plant parts and b) decanting the extract to obtain the composition; a method of maintaining hygiene in an oral cavity, said method comprising act of contacting the oral cavity with a composition comprising extracts of Emblica officinalis, Terminalia chebula, Terminalia bellerica, Glycyrrhiza glabra and Azadiracta indica; and a method of managing mucosal inflammatory conditions, said method comprising act of contacting the inflammated mucosa with a composition comprising extracts of Emblica officinalis, Terminalia chebula, Terminalia bellerica, Glycyrrhiza glabra and Azadiracta indica.

### DETAILED DESCRIPTION

The present disclosure relates to a composition comprising extracts of Emblica officinalis, Terminalia chebula, Terminalia bellerica, Glycyrrhiza glabra and Azadiracta indica. In an embodiment of the present disclosure, the extracts of the Emblica officinalis, the Terminalia chebula and the Terminalia bellerica are obtained from dried fruits of said species; and wherein the extract of the Glycyrrhiza glabra is obtained from root; and wherein the extract of the Azadiracta indica is obtained from combination of leaves and bark.

The present disclosure relates to a method for obtaining a composition comprising extracts of Emblica officinalis, Terminalia chebula, Terminalia bellerica, Glycyrrhiza glabra and Azadiracta indica, said method comprising acts of:
(a) soaking plant parts of Emblica officinalis, Terminalia chebula, Terminalia bellerica, Glycyrrhiza glabra and Azadiracta indica in a solvent to obtain extract of the plant parts; and
(b) decanting the extract to obtain the composition.

In an embodiment of the present disclosure, the plant parts are soaked for about a period ranging from about 8 hr to about 14 hr.

In another embodiment of the present disclosure, the concentration of each of the extracts is ranging from about 10% w/w to about 20% w/w.

In yet another embodiment of the present disclosure, the solvent is selected from a group comprising of water, ethyl alcohol, carboxymethyl cellulose, propylene glycol, and glycerine.

In another embodiment of the present disclosure, the plant parts are dried fruits of the Emblica officinalis, the Terminalia chebula and the Terminalia bellerica; root of the Glycyrrhiza glabra; and leaves and bark of the Azadiracta indica.

The present disclosure relates to a method of maintaining hygiene in an oral cavity, said method comprising act of contacting the oral cavity with a composition comprising extracts of Emblica officinalis, Terminalia chebula, Terminalia bellerica, Glycyrrhiza glabra and Azadiracta indica.

The present disclosure relates to a method of managing mucosal inflammatory conditions, said method comprising act of contacting the inflammated mucosa with a composition comprising extracts of Emblica officinalis, Terminalia chebula, Terminalia bellerica, Glycyrrhiza glabra and Azadiracta indica.

In an embodiment of the present disclosure, wherein the mucosal inflammatory conditions are selected from group comprising oral mucositis, oral submucous fibrosis and inflammations caused due to radiation in rectum or vagina.

In an embodiment of the present disclosure, the herbal formulation comprising extracts from five plants—Emblica officinalis, Terminalia chebula, Terminalia bellerica, Azadiracta indica, Glycyrrhiza glabra is found to be very effective in delaying the development of mucositis and its symptoms. This formulation is very cost effective and convenient to use. The cost of the current mouthwash is economical, along with having good analgesic, antibacterial, anti-inflammatory and mucolytic effect, when compared to those present in prior art such as the agent GMCSF and benzidamine. For example, the approximate cost of obtaining the current mouthwash in India is about INR 10.00 per day whereas the agent GMCSF costs INR 2000.00 per vial and benzidamine costs INR 28.00 per 120 ml bottle. Radiation induced oral mucositis also has a significant economic impact due to costs associated with pain management, antibiotic use, liquid diet supplements, gastrostomy tube placement and hospitalization. In one study of patients receiving radiotherapy for head and neck cancer, oral mucositis was associated with an increase in costs ranging from $1700-$6000 per patient, depending on the grade of oral mucositis. (Ref: Elting L S, Cooksely C D, Chambers M S, Garden A S. Risk, outcomes and costs of radiation—induced oral mucositis among patients with head and neck malignancies. Int J Radiat Oncol Biol Phys. 2007 Jul. 15; 68(4):1110-20).

The current mouth wash prevents development of mucositis in the initial phase of radiotherapy and minimizes the symptoms and signs of mucositis. It also minimizes the severity of mucositis. The current mouth wash controls mucositis and there by delays the occurrence of mucositis. Further, it can be used to control oral mucositis in a radiotherapy patient already having oral mucositis.

The composition in the current invention, significantly controlled the onset of radiation induced oral mucositis, and significantly reduced pain and consequent use of analgesics in patients. The current invention demonstrates good analgesic action which is a major problem for patients with mucositis. Even in patients who develop grade 3 mucositis, use of the current invention resulted in significant decrease in use of strong analgesics (step 3) p value<0.001. The analgesic effect is owed to the use of current mouthwash. If the ingredients in the mouth wash were singly used, significant analgesic effect will not be obtained. In addition to its anti-inflammatory effect, it also has a local anesthetic effect providing a synergistic mechanism for best relief. The mouth wash also demonstrated its efficacy in maintaining good oral hygiene thus demonstrating a possible antibacterial effect. This is also supported by the effect that the use of antibiotics to control secondary infections was much less in the group that used the mouthwash. Moreover, late side effects of radiotherapy, including dryness of mouth and intolerance to spicy food was much less in patients who used the mouthwash. (p value<0.008 & <0.001 respectively). This property is due to the ability of the mouthwash to protect the minor salivary glands from radiation induced damage. Such properties and associated benefits have not been reported for any of the components of the present composition, either individually or in combination with each other.

In an embodiment of the present disclosure, a herbal mouth wash composition comprises extracts from five plants—Emblica officinalis, Terminalia chebula, Terminalia bellerica, Azadiracta indica, and Glycyrrhiza glabra. The constituent of the extract are taken in quantities ranging from 10% w/w to about 20% w/w. The extracts of the Emblica officinalis, the Terminalia chebula and the Terminalia bellerica are extracts of dried fruit; the extract of the Glycyrrhiza glabra is extract of root and the extract of the Azadiracta indica is extract of both its leaves and bark. The concentration of the each of the extract in the composition is ranging from about 10% w/w to about 20% w/w.

It is preferred to get the extract of Azadiracta indica individually from its leaves and bark and then to combine them because the properties of leaves and bark are different. Neem leaves provide a cooling action for the mouth wash where as the bark has ulcer healing and anti-inflammatory property

In another embodiment of the present disclosure, the solvent is selected from a group comprising of water, ethyl alcohol, carboxymethyl cellulose, propylene glycol, and glycerine. Ethyl alcohol cannot be used as a solvent in patients undergoing radiotherapy as it can aggrevate mucositosis. However, it can be used as a solvent in normal individuals. The other solvents to be used include carboxymethyl cellulose, propylene glycol and glycerine to increase the viscosity of the current mouthwash so that it will remain more time in the oral cavity.

In another embodiment of the present disclosure, the herbal mouthwash delays the onset of oral mucositis during radiotherapy. The herbal mouthwash showed decreased requirement of analgesics to delay the onset of mucositis. The analgesic usage by patients using herbal mouth wash was significantly low (p value=0.0001). Patients requiring step 2 and step 3 analgesics were significantly high in the control group. The present mouthwash showed a mucolytic effect and helps to keep the mouth clean. Moreover, late side effects of radiotherapy in head and neck cancer, dryness of mouth (xerostomia) and intolerance to spicy food are less in those who used herbal mouthwash. No toxicity is noticed from the mouth wash and it does not adversely affect the treatment response as established from the results tabulated in Table 3.

In another embodiment of the present disclosure, the composition is tested in a group of patients undergoing radiation therapy against a group of control who used the standard practice of soda/saline mouthwash. The results also show that the mouthwash delays the onset of oral mucositis in patient undergoing radiation for oral cancer. The oral hygiene is good in these patients and the use for antibiotics was less. There is a significant decrease in the late side effects of radiation to the oral cavity such as dryness of mouth, intolerance to spicy food in the patients who use ayurvedic herbal mouthwash. The use of mouth wash did not affect the response to radiotherapy for these patients. The Tables 1-3 show that the composition of the present disclosure delays onset of oral mucositis. Table 4 shows the hemoglobin levels and weight changes of the patients before and after treatment with the composition.

The present disclosure is further elaborated by the following example. However it should not be construed to limit the scope of the disclosure.

### Example 1

50 g of the dried power comprising equal quantity (i.e. 16.67%) of the plant parts of Emblica officinalis, Terminalia chebula, Terminalia bellerica, Azadiracta indica (i.e., each for leaves and bark), Glycyrrhiza glabra are soaked in 300 ml of water and kept overnight. The supernatant is decanted out and used for cleaning the oral cavity.

### Example 2

50 g of the dried powder comprising the 19% w/w of the plant part of Emblica officinalis, 20% w/w of that of Terminalia chebula, 20% w/w of Terminalia bellerica, 21% w/w of Azadiracta indica, 20% w/w of Glycyrrhiza glabra are soaked in 300 ml of water and kept overnight. The supernatant is decanted out and used for cleaning the oral cavity.

### Example 3

50 g of the dried power comprising the 20% w/w of the plant part of Emblica officinalis, 18% w/w of that of Terminalia chebula, 19% w/w of Terminalia bellerica, 24% w/w of Azadiracta indica (Bark 12% and leaves 12%), 19% w/w of Glycyrrhiza glabra are soaked in 300 ml of water and kept overnight. The supernatant is decanted out and used for cleaning the oral cavity.

### Example 4

The composition is tested in 75 subjects against 73 controls who used the standard practice of soda/saline mouthwash.

300 ml of the oral mouth wash composition is used in parts by the cancer patients to rinse their mouth four times a day. On day 8, 65.3% of the patients who are using the oral mouth wash, did not exhibit mucositis. However, only 27.8% of the control patients are free of mucositis. The p-value being <0.001. Similar results are observed on day 15 (Table 1). RTOG grading of mucositis was used to assess the grade of mucositis experienced by patients in both arms. According to RTOG grading of mucositis:

Grade 0=No change over baseline

### Grade 1=Erythema

Grade 2=Patchy mucositis which may produce an inflammamtory serosanguinitis discharge Grade 3=Confluent fibrinous mucositis
Grade 4=Ulceration, hemorrhage or necrosis.

Patients using the mouth wash showed reduced pain. Pain was assessed using a Visual Analogue Scale.] Visual Analogue Scale indicate the intensity of pain in general ranging from 0 to 10, zero being the minimum and 10 being the maximum. We have grouped 0-3 as mild pain, 4-7 as moderate pain and 8-10 as severe pain. The requirement of analgesics by patients under the effect of the oral mouth wash composition was also reduced. The use of analgesics by patients undergoing radiotherapy for head and neck cancer were assessed on the basis of WHO step ladder analgesic requirement. Zero means no analgesics were used. 1=Oral non-opioid, non-steroidal anti-inflammatory drug (NSAID) and is used in mild pain and is simple. 2=NSAID+weak opioid and is used in moderate pain. 3=Strong opioid and is used in severe pain. The percentage of patients who used step 2 and step 3 analgesics in the intervention arm was significantly low p value being <0.001. On day 22, 91.9% of patients using the mouth wash did not require administration of antibiotics. It was only 64.3% in control patients. Thus, the use of antibiotic is less in patients using the instant oral mouth wash. Oral hygiene of 37.8% of patients using the mouth wash was good on day 22, while it was only 10% in control patients. (Table 1)

Thus, onset of mucositis is delayed in patients using the herbal mouthwash. Oral hygiene was significantly better in patients using the herbal mouthwash. The requirements of antibiotic and analgesics are significantly lower in patients who use the herbal mouth wash.

TABLE 1
Table showing severity of symptoms and oral hygiene in patients during radiotherapy treatment in both arms.

| | Day 0 | | Day 8 | | Day 15 | | Day 22 | |
|---|---|---|---|---|---|---|---|---|
| | Intervention (%) | Control (%) | Intervention (%) | Control (%) | Intervention (%) | Control (%) | Intervention (%) | Control (%) |
| Number | 75 | 72 | 75 | 72 | 75 | 71 | 74 | 70 |
| Mucositis | | | | | | | | |
| Grade 0 | | | 49 (65.3) | 20 (27.8) | 3 (4.0) | 0 (0.0) | | |
| Grade 1 | | | 26 (34.7) | 43 (59.7) | 14 (18.7) | 5 (7.0) | | |
| Grade 2 | | | 0 | 9 (12.5) | 43 (57.3) | 40 (56.3) | 8 (10.8) | 4 (5.7) |
| Grade 3 | | | | | 15 (20.0) | 26 (36.6) | 66 (89.2) | 66 (94.3) |
| P value | | | <0.001 | | <0.017 | | 0.269 | |
| Pain | | | | | | | | |
| 0-3(mild) | 70 (93.3) | 42 (58.3) | 70 (93.3) | 42 (58.3) | 31 (41.3) | 9 (12.7) | 44 (59.5) | 14 (20.0) |
| 4-7(moderate) | 4 (5.3) | 27 (37.5) | 4 (5.3) | 27 (37.5) | 43 (57.3) | 38 (53.5) | 26 (35.1) | 24 (34.3) |
| 8-10(severe) | 1 (1.3) | 3 (4.2) | 1 (1.3) | 3 (4.2) | 1 (1.3) | 24 (33.8) | 4 (5.4) | 32 (45.7) |
| P value | 0.077 | | <0.001 | | <0.001 | | <0.001 | |
| Oral hygiene | | | | | | | | |
| Poor | 1 (1.3) | 0 | 2 (2.7) | 6 (8.3) | 6 (8.0) | 12 (16.9) | 8 (10.8) | 32 (45.7) |
| Fair | 10 (13.3) | 4 (5.6) | 7 (9.3) | 26 (36.1) | 18 (24.0) | 43 (60.6) | 38 (51.4) | 31 (44.3) |
| Good | 64 (85.3) | 68 (94.4) | 66 (88.0) | 40 (55.6) | 51 (68.0) | 16 (22.5) | 28 (37.8) | 7 (10.0) |
| P value | 0.163 | | <0.001 | | <0.001 | | <0.001 | |

TABLE 2
Table showing the requirement of supportive drugs in patients during radiotherapy in both arms.

| | Day 0 | | Day 8 | | Day 15 | | Day 22 | |
|---|---|---|---|---|---|---|---|---|
| | Intervention (%) | Control (%) | Intervention (%) | Control (%) | Intervention (%) | Control (%) | Intervention (%) | Control (%) |
| Analgesics* | | | | | | | | |
| 0 | 64 (85.3) | 59 (81.9) | 69 (92.0) | 38 (52.8) | 37 (49.3) | 6 (8.5) | 21 (28.4) | 3 (4.3) |
| 1 | 10 (13.3) | 12 (16.7) | 6 (8.0) | 30 (41.7) | 35 (46.7) | 55 (77.5) | 46 (62.2) | 50 (71.4) |
| 2 | 1 (1.3) | 1 (1.4) | 0 | 4 (5.6) | 3 (4.0) | 8 (11.3) | 6 (8.1) | 15 (21.4) |
| 3 | | | | | 0 | 2 (2.8) | 1 (1.4) | 2 (2.9) |
| P value | 0.85 | | <0.001 | | <0.001 | | <0.001 | |
| Antibiotics | | | | | | | | |
| No | | | 75 (100.0) | 65 (90.3) | 68 (90.7) | 52 (73.2) | 68 (91.9) | 45 (64.3) |
| Yes | | | 0 | 7 (9.7) | 7 (9.3) | 19 (26.8) | 6 (8.1) | 25 (35.7) |
| P value | | | 0.006 | | 0.006 | | <0.001 | |

*Analgesics 0 = No analgesics, 1 = Step 1 analgesics- Non Steroidal Anti Inflammatory Drugs (NSAID), 2 = Step 2 analgesics- NSAID + weak opioid, 3 = Step 3 analgesics Opioids

### Example 5

The response to radiotherapy for the primary cancer was assessed CR=Complete Response to radiotherapy (Complete disappearance of the disease), PR=Partial Response to radiotherapy (More than 50% regression of the disease), PD=Progressive Disease. 92.6% of the patients using the oral mouth wash were responsive to radiation therapy. This means that the use of mouth wash did not affect the response to radiotherapy while minimizing the side effect that is oral mucositis. 73.5% of patients did not show dryness of mouth, while it was only 56.5% in control patients. Only 19.1% of patients showed intolerance to spicy food while it was 79% in control patients. (Table 3)

Thus, the use of mouth wash did not affect the response to radiotherapy. Moreover; the late side effects of radiotherapy such as dryness of mouth and intolerance to spicy food were significantly reduced in patients in the intervention arm.

TABLE 3
Comparison of response to treatment and late toxicity in both arms

| | | |
|---|---|---|
| Intervention | | |
| (%) | Control (%) | p-value |

| | | |
|---|---|---|
| Number | 68 | 62 |

| | | | | | |
|---|---|---|---|---|---|
| Response to treatment | | | | | |
| CR¹ | 63 | (92.6) | 49 | (79.0) | 0.068 |
| PR² | 5 | (7.4) | 12 | (19.4) | |

| | | | |
|---|---|---|---|
| PD³ | 0 | 1 | (1.6) |

| | | | | | |
|---|---|---|---|---|---|
| Dryness of the mouth | | | | | |
| No | 50 | (73.5) | 35 | (56.5) | 0.008 |
| Mild | 16 | (23.5) | 13 | (21.0) | |
| Moderate | 2 | (2.9) | 13 | (21.0) | |

| | | | |
|---|---|---|---|
| Severe | 0 | 1 | (1.6) |

| | | | | | |
|---|---|---|---|---|---|
| Intolerance | | | | | |
| No | 8 | (11.8) | 3 | (4.8) | <0.001 |
| Mild | 27 | (39.7) | 6 | (9.7) | |
| Moderate | 20 | (29.4) | 4 | (6.5) | |
| Severe | 13 | (19.1) | 49 | (79.0) | |

¹= Complete Response;
²= Partial Response;
³= Progressive Disease

### Example 6

The hemoglobin levels of patients remain the same before and after using the mouthwash with change in p-value being 0.09. Same is the case with respect to the weight of the patients with change in p-value being 0.29. (Table 4)

TABLE 4
Comparison of mean Hb levels and weight change of the
patients before and after treatment in both arms.

| | | |
|---|---|---|
| Intervention | Control | |
| Mean (SD) | Mean (SD) | p-value |

| | | | | | |
|---|---|---|---|---|---|
| Hb | | | | | |
| Before | 13.0 | (1.8) | 12.9 | (1.5) | 0.89 |
| After | 13.1 | (1.6) | 12.6 | (1.6) | 0.08 |
| Change | 0.1 | (1.3) | −0.3 | (1.7) | 0.09 |
| Weight | | | | | |
| Before | 47.5 | (9.5) | 48.6 | (10.2) | 0.52 |
| After | 45.3 | (10.4) | 45.7 | (11.5) | 0.85 |
| Change | −2.2 | (3.3) | −2.9 | (4.6) | 0.29 |

The instant disclosure thus deals with an herbal oral mouth wash composition comprising extracts of Emblica officinalis, Terminalia chebula, Terminalia bellerica, Glycyrrhiza glabra and Azadiracta indica. The mouth wash has the property of delaying the onset of oral mucositis, reducing the dryness of the mouth and intolerance to spicy food, in patients.

### Example 7

Patients undergoing radiation in the intervention arm though had delayed onset of mucositis, 89.2% of patients had grade 3 mucositis, towards the end of radiotherapy (day 22). Pain is mainly due to the inflammation. Even though patients in the intervention arm had grade 3 mucositis, the intensity of pain and analgesic requirement was significantly low in patients in the intervention arm towards the end of radiation (Table 5).

TABLE 5
Comparison of the local anesthetic effect of the mouthwash
in patients before and after treatment in both arms.

| | Intervention arm | Control arm |
|---|---|---|
| Grade 3 mucositis | 89.2% | 94.3% |
| Pain score 8-10 | 5.4% | 45.7% |
| Step 2 analgesic | 8.1% | 21.4% |
| Step 3 analgesic | 1.4% | 2.9% |

### Example 8

A clinical trial with the prescribed mouthwash, was done as a pilot study with 8 patients. These 8 patients were divided into two groups of four. In one group (Group A) the prescribed mouthwash in which all the 6 ingredients are taken in equal quantity ie, (16.67% each). In the other group (Group B) we had doubled the concentration of Glycyrrhiza glabra (33.34%) and the rest of the five ingredients were taken in equal quantities (13.5% each). Both group of patients were given the same dose of radiation and followed up for four weeks. All the four patients in Group A had delayed onset of mucositis, less pain, low use of analgesics and antibiotics and had good oral hygiene. In group B one patient discontinued the radiotherapy treatment. The rest three patients had to use analgesics. If the above said actions such as analgesic, antiinflammatory, local anaesthetic effect of the mouthwash are due to Glycyrrhiza glabra alone, these effects should have been enhanced in patients who used the mouthwash Group B. This clearly shows that the analgesic, antiinflammatory, local anaesthetic effect and the property to delay the onset of mucositis of the mouthwash is due to a synergistic action of Glycyrrhiza glabra with other five ingredients.

In group B, the proportion of Glycyrrhiza glabra is doubled (33.3%) than that in Group A (16.67%). However, the compliance to Group B is only 50% (2 out of 4) without any added benefit. Dryness, coating of tongue and burning sensation is experienced in 50% of patients who used Group B mouthwash as against none in Group A. Two out of four patients on Group B mouthwash developed mucositis towards the end of second week of radiation while two out of four patients on Group A mouthwash developed mucositis towards the end of third week. Tolerance to the mouthwash is excellent in Group A with lower percentage of Glycyrrhiza glabra. The proportions of various compounds used in Group A is optimum with good synergy.

In combination B, the proportion of Glycyrrhiza glabra is double (33.3%) than that in Group A (16.67%).

### Patient 1: Combination: B, Patient Underwent Radiation for Carcinoma Tongue

5 Oct. 2004—(after 6 radiations)—patient has burning sensation in mouth
11 Oct. 2004—(after 8 radiations) has pain and discomfort, Grade 1 mucositis present
13 Oct. 2004—(after 13 radiations) patient has pain while using the mouthwash, poor oral hygiene, indication for analgesics and antibiotics, super added infection present. As there was no added benefit using combination B we stopped the B combination to patient 1 and the patient was given combination A thereafter.
15 Oct. 2004: (after 15 radiations)-burning sensation relieved using A group with mouthwash
20 Oct. 2004: (after 18 radiations) patient feeling good with combination A
23 Oct. 2004: (after 20 fractions; end of radiation) mucositis and infection controlled, there is only slight burning sensation in the oral cavity.

### Patient 2: Combination Used: B; Patient Underwent Radiation for Carcinoma Tongue

5 Oct. 2004—(after 5 fractions)—no discomfort
8 Oct. 2004—(after 8 fractions)—salivation present
12 Oct. 2004 (after 10 fractions)—coating over the mouth, dryness present
14 Oct. 2004 (after 13 fractions)—severe pain present
15 Oct. 2004—discontinued radiation

### Patient 3: Combination Used: B; Patient Underwent Radiation for Carcinoma Tongue

8 Oct. 2004—(after 8 fractions of radiation) dryness of mouth
14 Oct. 2004—(after 11 radiations)—pain present
15 Oct. 2004—(after 12 radiations)—saliva thickening present
20 Oct. 2004—(after 15 radiations end of radiation)—mucositis present, no taste sensation, tongue coated

### Patient 4: Combination Used: B; Patient Underwent Radiation for Carcinoma Tongue

19 Oct. 2004—(after 8 radiations)—no discomforts
29 Oct. 2004—(after 14 radiations) coated tongue, infection present, no taste sensation
5 Nov. 2004—(after 19 fractions)—coated tongue, infection present, no taste sensation
8 Nov. 2004—(after 20 fractions; end of radiation)—unable to take solid food, coated tongue, general weakness

### Patient 1 Combination Used: A; Patient Underwent Radiation for Carcinoma Tongue

6 Oct. 2004—(after 10 radiations)—no discomfort
15 Oct. 2004—(after 15 radiations)—cooling effect inside the oral cavity
16 Oct. 2004—(after 18 radiations)—grade 3 mucositis present, no pain, not on analgesics, oral hygiene good.
20 Oct. 2004—(after 20 radiations, end of radiation)—mucositis under control

### Patient 2. Combination Used: A; Patient Underwent Radiation for Carcinoma Tongue

12 Oct. 2004—(after 8 radiations)—no mucositis, no dryness, no burning sensation
25 Oct. 2004—(after 15 radiations)—no pain, no mucositis, patient not on analgesics
3 Nov. 2004—(after 20 fractions, end of radiation)—reaction mild, no discomfort

### Patient 3. Combination Used: A; Patient Underwent Radiation for Carcinoma Tongue

11 Oct. 2004—after 8 radiations—pt having cooling effect, Salivation improved
15 Oct. 2004—after 12 radiations—no discomfort, grade 1 mucositis
20 Oct. 2004—after 15 radiations—slight pain, no discomfort
28 Oct. 2004—after 20 radiations; end of radiation—no infection

### Patient 4. Combination Used: A; Patient Underwent Radiation for Carcinoma Buccal Mucosa

11 Oct. 2004—after 5 radiations—no pain, no reaction
21 Oct. 2004—after 12 radiations—no pain, no reaction, no infection, can take spicy food
26 Oct. 2004—after 15 radiations; end of radiation grade 2 mucositis, no pain, no infection

We claim:

1. A composition comprising extracts of Emblica officinalis, Terminalia chebula, Terminalia bellerica, Glycyrrhiza glabra and Azadiracta indica.

2. The composition as claimed in claim 1, wherein the extracts of the Emblica officinalis, the Terminalia chebula and the Terminalia bellerica are obtained from dried fruits of said species; and wherein the extract of the Glycyrrhiza glabra is obtained from root; and wherein the extract of the Azadiracta indica is obtained from combination of leaves and bark.

3. A method for obtaining a composition comprising extracts of Emblica officinalis, Terminalia chebula, Terminalia bellerica, Glycyrrhiza glabra and Azadiracta indica, said method comprising acts of:
(a) soaking plant parts of Emblica officinalis, Terminalia chebula, Terminalia bellerica, Glycyrrhiza glabra and Azadiracta indica in a solvent to obtain extract of the plant parts; and
(b) decanting the extract to obtain the composition.

4. The method as claimed in claim 3, wherein the plant parts are soaked for about a period ranging from about 8 hr to about 14 hr.

5. The method as claimed in claim 3, wherein the concentration of each of the extracts is ranging from about 10% w/w to about 20% w/w.

6. The method as claimed in claim 3, wherein the solvent is selected from a group comprising of water, ethyl alcohol, carboxymethyl cellulose, propylene glycol, and glycerine.

7. The method as claimed in claim 3, wherein the plant parts are dried fruits of the Emblica officinalis, the Terminalia chebula and the Terminalia bellerica; root of the Glycyrrhiza glabra; and leaves and bark of the Azadiracta indica.

8. A method of maintaining hygiene in an oral cavity, said method comprising act of contacting the oral cavity with a composition comprising extracts of Emblica officinalis, Terminalia chebula, Terminalia bellerica, Glycyrrhiza glabra and Azadiracta indica.

9. A method of managing mucosal inflammatory conditions, said method comprising act of contacting the inflammated mucosa with a composition comprising extracts of Emblica officinalis, Terminalia chebula, Terminalia bellerica, Glycyrrhiza glabra and Azadiracta indica.

10. The method as claimed in claim 9, wherein the mucosal inflammatory conditions are selected from group comprising oral mucositis, oral submucous fibrosis and inflammations caused due to radiation in rectum or vagina.
